(19) United States
(12) Patent Application Publication
(10) Pub. No.: US 2006/0036215 A1
(43) Pub. Date: Feb. 16, 2006

(54) Surgical apparatus

(76) Inventor: Mikhail Boukhny, Laguna Niguel, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/916,674

(22) Filed: Aug. 12, 2004

(51) Int. Cl. A61M 5/32

(52) U.S. Cl. 604/175

(57) ABSTRACT

A sealant ring that fits around the shaft of the manipulation tool and seals the shaft so as to reduce or prevent the flow of irrigating fluid out of the eye from the wound around the shaft. The seal also helps prevent collapse of the would about the shaft of the manipulation tool. In addition, an adjustable stop might be used of similar construction as the seal. the stopper can be used to help force the seal into the wound.

### BACKGROUND OF THE INVENTION

This invention relates generally to the field of cataract surgery and more particularly to an apparatus for cataract phacoemulsification surgery.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light that can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. A typical surgical handpiece suitable for phacoemulsification procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve.

Recently, a modified phacoemulsification technique called “bimanual” phacoemulsification has been adopted by many surgeons. With the bimanual technique, the irrigation sleeve is removed from around the ultrasonically drive tip. This allows for the small tip to be inserted into the eye through a smaller incision. Irrigation fluid is supplied by a second irrigating tip. The second tip may include a manipulation tool. Additional information concerning traditional phacoemulsification and bimanual phacoemulsification is included in U.S. patent Publication No. US 2003/0069594 A1. And in particular, Paragraphs [0036] through [0037] and FIGS. 6-8, which are incorporated herein by reference. As described in this reference, the second instrument does not use an outer silicone infusion sleeve. Rather the shaft of the manipulation tool is either hollow with irrigation ports, or solid with a separate hollow irrigating conduit containing irrigation ports. Without the outer silicone sleeve, sealing of the wound is minimal. This allows excessive irrigating fluid to escape out of the eye through the wound. Excessive wound leakage can cause shallowing of the anterior chamber, excessive turbulence and premature removal of the protective viscoelastic material. Excessive wound leakage can also cause over-hydration of the wound tissue, possibly resulting in edema.

Therefore, a need continues to exist for a device to seal the shaft of the manipulation tool.

### BRIEF SUMMARY OF THE INVENTION

The present invention improves upon the prior art by providing a sealant ring that fits around the shaft of the manipulation tool and seals the shaft so as to reduce or prevent the flow of irrigating fluid out of the eye from the wound around the shaft. The seal also helps prevent collapse of the would about the shaft of the manipulation tool. In addition, an adjustable stop might be used of similar construction as the seal. the stopper can be used to help force the seal into the wound.

Accordingly, one objective of the present invention is to provide a device for reducing the flow of irrigating fluid out of the eye through the wound around a manipulation device.

Another objective of the present invention is to provide a device for preventing the collapse of a surgical wound around a manipulation tool.

These and other advantages and objectives of the present invention will become apparent from the detailed description and claims that follow.

### BRIEF DESCRIPTION OF THE DRAWINGS

FIG. 1 is a partial side elevational view of a surgical manipulation tool that may be used with the present invention.

FIG. 2 is a partial cross-sectional view of a surgical manipulation tool inserted into a surgical wound.

FIG. 3 is a partial cross-sectional view of a surgical manipulation tool with the seal and the stopper of the present invention installed on the shaft.

FIG. 4 is an end elevational view of a first embodiment of the seal of the present invention.

FIG. 5 is an end elevational view of a second embodiment of the seal of the present invention.

FIG. 6 is a side elevational view of the stopper of the present invention.

FIG. 7 is an end elevational view of the stopper of the present invention.

### DETAILED DESCRIPTION OF THE INVENTION

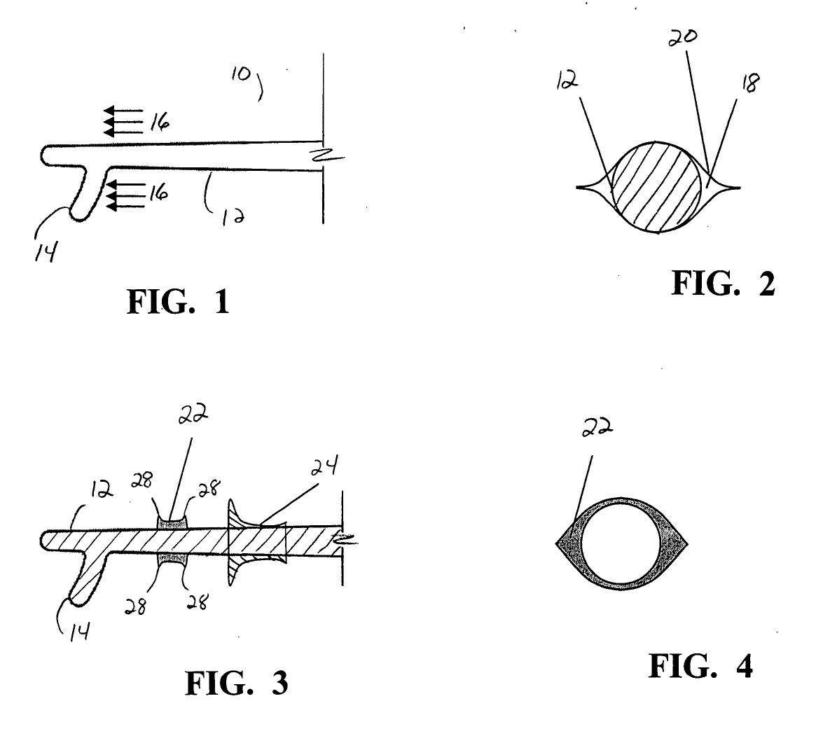

As best seen in FIGS. 1 and 2, prior art manipulation tool 10 generally include shaft 12 and hook 14. Alternatively, a chopper, spatula or other suitable tool may be used in place of hook 14. Relatively thin shaft 12 allows for gap 18 between shaft 12 and wound 20, thereby allows excessive flow 16 hook out of wound 20.

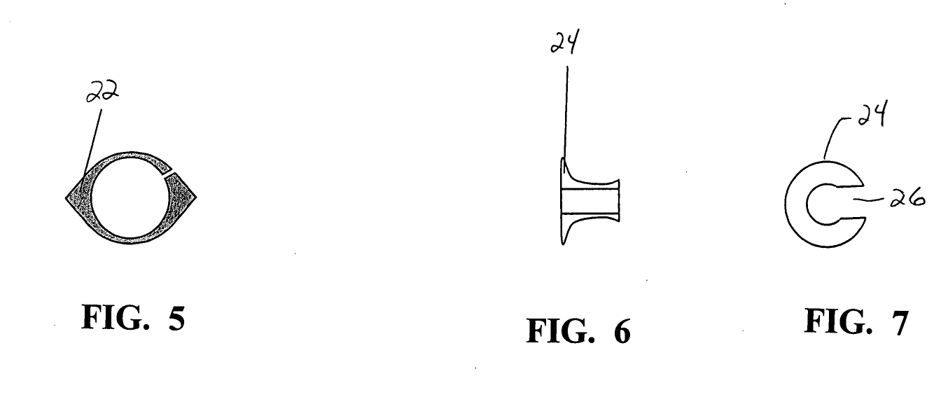

As seen in FIGS. 3, 4 and 5, seal 22 of the present invention fits around shaft 12 and is sized and shaped to approximate the size and shape of wound 20. Seal 22 preferably is approximately 1.5 or 2.0 millimeters long if a clear cornea incision is being used and approximately 3.0 to 4.0 millimeters long if a sclera tunnel incision is being used. Seal 22 preferably is made from a biocompatible, soft elastomeric material such as silicone rubber, and may be made as a solid ring in cross-section, as shown in FIG. 4, or as a split ring in cross-section, as shown in FIG. 5 to aid in the installation of seal 22 on shaft 12 of manipulation tool 10. Seal 22 may also have lips 28 to aid in holding seal 22 in wound 20 and minimize the possibility of inadvertent dislodging of seal 22 from wound 20 during manipulations of tool 10.

As seen in FIGS. 3, 6 and 7, stopper 24 may also be used with the present invention. Stopper 24 is made from a relatively firm thermoplastic or other suitable material and is sized and shaped to slide relatively easily on shaft 12, as opposed to seal 22 which is relatively resistant to sliding on shaft 12. As seen in FIGS. 6 and 7, stopper 24 has open side 26 allowing stopper 24 to be snapped in place on shaft 12. Stopper 24 may pushed distally down shaft 12 to help push seal 22 into wound 20, and removed from shaft 12 or be withdrawn proximally down shaft 12 and away from wound 20 when not needed. One skilled in the art will recognize that as shaft 12 is tapered distally toward hook 14, movement of seal 22 and stopper 24 is more easily accomplished in a distal direction than in a proximal direction.

This description is given for purposes of illustration and explanation. It will be apparent to those skilled in the relevant art that changes and modifications may be made to the invention described above without departing from its scope or spirit.

I claim:

1. A surgical apparatus, comprising:
a) a surgical manipulation tool having a shaft; and
b) a seal sized and shaped to fit over the shaft.

2. The surgical apparatus of claim 1 wherein the seal is made from a relatively soft elastomer.

3. The surgical apparatus of claim 1 wherein the seal has a plurality of retaining lips to assist in retaining the seal in a surgical wound.

4. The surgical apparatus of claim 1 further comprising a stopper sized and shaped to fit over the shaft.

5. A surgical apparatus, comprising:
a) a surgical manipulation tool having a shaft;
b) a seal sized and shaped to fit over the shaft, the seal having a plurality of retaining lips to assist in retaining the seal in a surgical wound; and
c) a stopper sized and shaped to fit over the shaft.
